(19) United States
(12) Patent Application Publication
(10) Pub. No.: US 2023/0194559 A1
(43) Pub. Date: Jun. 22, 2023

(54) DEVICE FOR MEASURING INSTANTANEOUS SPRINT VELOCITY

(71) Applicant: ECOLE POLYTECHNIQUE FEDERALE DE LAUSANNE (EPFL), Lausanne (CH)

(72) Inventors: Salil APTE, Lausanne (CH); Kamiar AMINIAN, La Tour-de-Peilz (CH); Farzin DADASHI, Pully (CH); Benoît MARIANI, Blonay (CH)

(21) Appl. No.: 17/924,977

(22) PCT Filed: May 12, 2021
(86) PCT No.: PCT/IB2021/054045

§ 371 Date: Nov. 11, 2022

(30) Foreign Application Priority Data
May 14, 2020 (WO) PCT/IB2020/054572

(51) Int. Cl. G01P 3/00; A63B 69/00

(52) U.S. Cl. CPC G01P 3/00; A63B 69/0028; A63B 2220/30; A63B 2220/836; A63B 2220/40

(57) ABSTRACT

The invention concerns a device for measuring instantaneous sprint velocity, said device consisting of at least one position and/or one velocity sensor and one IMU sensor that respectively provide position and/or velocity and acceleration signals and wherein said signals are fused. The invention also concerns a method for measuring instantaneous sprint velocity comprising the use of at least one position and/or one velocity sensor and one IMU sensor that respectively provide position and/or velocity and acceleration signals and wherein said signals are fused.

### FIELD OF INVENTION

The present invention relates to the velocity measurement of athletes and more precisely sprint velocity.

### STATE OF THE ART

Functional tests based on rapid movements over short distances, like the sprint test and the T-test for agility, are commonly utilized for measuring the functional capacity of athletes for multiple sports like soccer, hockey, rugby, athletics, etc. Velocity profile obtained during the sprint test is used to create the horizontal force-velocity (F-V) and horizontal power-velocity (P-V) plots for athletes. These profiles, in addition to the spatiotemporal parameters and timings of the T-test, are crucial for designing personalized training programs and evaluating injury risks (Morin, J. B., & Samozino, P. (2016). Interpreting power-force-velocity profiles for individualized and specific training. International journal of sports physiology and performance, 11(2), 267-272). Predominant method of estimating the instantaneous velocity (Samozino, P., Rabita, G., Dorel, S., Slawinski, J., Peyrot, N., Saez de Villarreal, E., & Morin, J. B. (2016). A simple method for measuring power, force, velocity properties, and mechanical effectiveness in sprint running. Scandinavian journal of medicine & science in sports, 26(6), 648-658) is based on the assumption that the velocity profile (v_{mdl}) shows a first-order exponential behavior towards reaching the maximum velocity (vₘₐₓ) over time (t)

$${v}_{mdl} ( t ) = {v}_{max} ( 1 - {e}^{\{ - \frac{t}{τ} \}} ) \qquad ( 1 )$$

The obtained velocity profile (v_{mdl}) is differentiated to obtain horizontal acceleration, and subsequently the F-V and P-V profiles. While this method provides ease of use, it is imprecise since the sprint velocity profile for all athletes does not necessarily show an exponential behavior. Furthermore, this profile may not hold true for all sprinters across different sprint distances; the sprinters may not achieve maximum velocity over short distances such as 30 m or they may not be able to maintain maximum velocity over longer distances such as 60 to 100 m. Sprint velocity has also been estimated with an application for a smartphone (Stanton 2016); wherein the in-built camera tracks and records the motion. Based on the distance entered manually, the application calculates the total sprint time and subsequently the mean velocity. Thus, this application cannot estimate instantaneous velocity and the measurable sprint distance might be limited by the field-of-view of the camera.

Recently, a magnetic and inertial measurement unit (MIMU) based algorithm (Setuain 2018) has been developed to assess sprint mechanics with various parameters such as maximal velocity, maximal horizontal force and power, velocity at zero horizontal force, etc., for 20 m sprints. Though this work allows the measurement of sprint mechanics using a single MIMU mounted on the trunk, the algorithm assumes a first-order exponential behaviour and relies on the use of split times from photocells at specific distances to estimate the parameters of the exponential equation. Other works on velocity estimation using a trunk-based MIMU (R. D. Gurchiek 2018, R. D. Gurchiek 2019), also assumed the first-order exponential behaviour for drift removal. Nevertheless, as explained previously, this behaviour may not hold true over different sprint distances and sub-maximal efforts. Finally, the ground velocity signal from GNSS alone is not responsive enough to measure the velocity during sprint (Nagahara 2017) and can lead to an underestimation of the sprint velocity. This issue is even more exacerbated among elite athletes, who produce a high magnitude of horizontal acceleration and for whom, the timing difference can be critical (J.-B. a. Morin 2016).

### GENERAL DESCRIPTION OF THE INVENTION

The proposed invention solves the problems mentioned in the previous chapter by combining the signals from position and/or velocity and IMU sensors (sensor fusion approach). This combination notably improves the sprint velocity measurement because the position or velocity sensor alone would not be adequately responsive to a high acceleration such as the one that may occur during a sprint. In case of indoor environments, wherein a GNSS sensor is used and wherein GNSS signals are not accessible, this algorithm can also be extended to use signals from other position and/or velocity sensing devices such as ultra-wideband (UWB) receivers and photoelectric sensors. While these sensors are likely to be non-wearable, the said indoor environments afford the use of such sensors without compromising utility. Furthermore, the proposed invention may provide the spatiotemporal parameters for the sprint, such as the time for which the foot is in contact with the ground on every step and the step length. These parameters, along with the force-power-velocity profiles can provide a comprehensive tool for monitoring athlete capacity and condition, which can be further used for evaluating athlete readiness after injury and for designing optimal training regimes.

The invention more precisely concerns a device for measuring instantaneous sprint velocity, said device consisting of at least one position and/or one velocity sensor and one IMU sensor that respectively provide position and/or velocity and acceleration signals and wherein said signals are fused.

According to a preferred embodiment the device consists of one GNSS sensor and one IMU sensor that respectively provide velocity and acceleration signals and wherein said signals are fused according to a Kalman filter.

The invention also includes a method for measuring instantaneous sprint velocity comprising the use of at least one position and/or one velocity sensor and one IMU sensor that respectively provide position and/or velocity and acceleration signals and wherein said signals are fused.

Preferably the method comprises the use of one wearable GNSS sensor that provides a velocity signal and one wearable IMU sensor that provides an acceleration signal, wherein said signals are fused according to a Kalman filter.

Advantageously a gradient descent algorithm is used as an orientation filter, for instance a Madwick filter (Madgwick, S. O., Harrison, A. J., & Vaidyanathan, R. (2011, June)), in combination with said Kalman filter. Following this approach offers the possibility to apply a linear one-dimensional model, thus allowing the use of a simple Kalman filter as velocity/acceleration filter to finally estimate the velocity during sprinting.

The orientation filter preferably utilizes the IMU sensor data to convert the acceleration signals from the sensor frame to the global frame, which is then given as input to said Kalman filter for estimating the precise sprint duration.

According to another embodiment, time-frequency analysis and machine learning techniques are used with the global-frame accelerometer signals and the estimated velocity data to compute the spatiotemporal parameters for the sprint.

The present invention, in particular its related algorithm, can be easily implemented on a wearable GNSS-IMU sensor, which is already widely used, for instance by soccer teams. This can enable a smoother product adaptation process with respect to existing products. Further, the simplicity of the algorithm results in low computational cost, thereby allowing a real-time implementation if necessary.

### DETAILED DESCRIPTION OF THE INVENTION

The invention will be better understood below, with non-limiting examples, together with some figures.

### BRIEF DESCRIPTION OF THE FIGURES

FIG. 1 shows different possibilities to position a device according to the invention on a runner.

FIG. 2 shows three phases of a sprinting task of a runner wearing on its back a device according to the invention.

FIG. 3 represents different measurements of the instantaneous velocity.

FIG. 4 shows different approximations of the instantaneous velocity profile.

FIG. 5 illustrates a flowchart of an algorithm used to estimate the instantaneous sprint velocity.

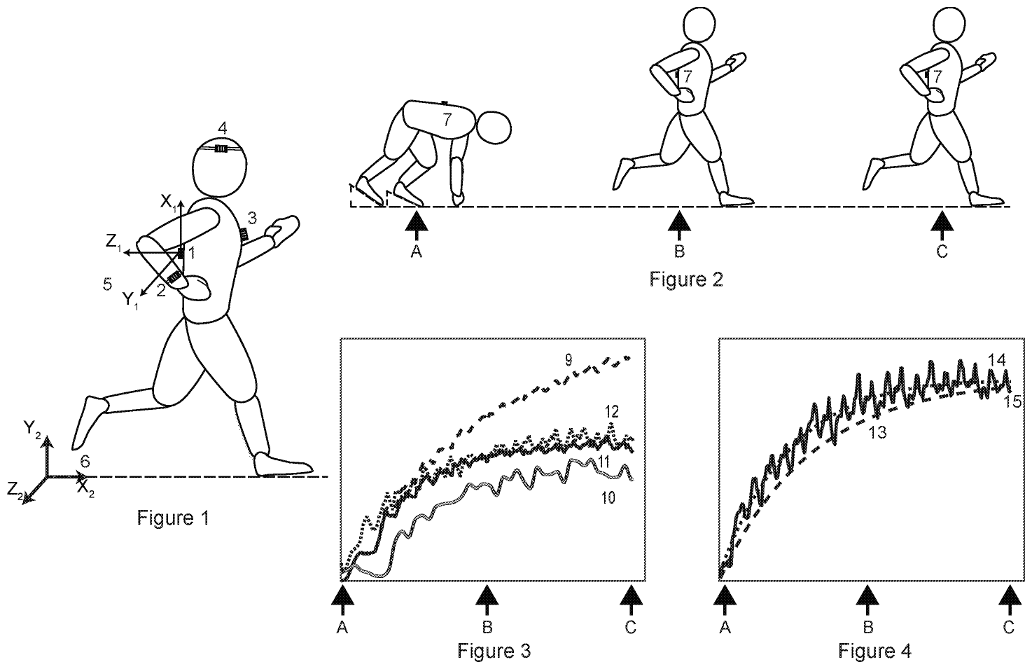

As shown on FIG. 1 the device according to the invention can be fixed to different parts of the runner, namely to the back of the trunk 1, to the wrist 2, to the front of the trunk 3 or to the head 4. The preferred position for the device is on the back of the trunk 1. FIG. 1 also shows a runner frame of reference 5 and a global frame of reference 6 in which the runner is moving.

FIG. 2 represents a runner with a device according to the invention 7 in different stages of a sprint, namely a start stage A, a middle stage B and an end stage C.

Measurements were conducted with nine healthy elite-level sprinters, four (3 male, 1 female, 60 m sprint time 7.49±0.35 s) at the Aix-les-Bains Athletics club and five (4 male, 1 female, 60 m sprint time 7.65±0.67 s) from the Lausanne Athletics club respectively. The Aix-les-Bains cohort performed 2×40 m and 2×60 m sprints, while the Lausanne one performed 2×30 m and 2×60 m sprints. These distances are typically used in sprint tests and for training sprinters. For both measurements, participants were wearing a vest equipped with a GNSS-IMU sensor (Fieldwiz, ASI, CH,) on the back of the trunk 1. Apart from the vest, the sprinters dressed as they would for a regular training session.

This GNSS-IMU wearable sensor was chosen because it is already used in soccer training for performance and training monitoring. This sensor, with a sampling frequency of 200 Hz for the IMU and 10 Hz for the GNSS unit, was used in the ‘airborne<4 g’ configuration of the in-built u-blox GNSS module. A speed radar (ATS Pro II, Stalker Sport, USA) with a sampling frequency of 50 Hz was positioned directly behind the starting point of the sprinter. Data from the radar was used in the measurements as a reference value for velocity. Photocells (Witty, Microgate corp, Italy) from the respective athletics clubs were used at the start and the end as reference value for the duration of the sprints.

FIG. 3 represents the instantaneous sprint velocity, as estimated from only one IMU sensor 9 or only one GNSS sensor 10. The instantaneous velocity from fusing the velocity and acceleration signals 11 aligns well with the reference instantaneous velocity signal 12.

FIG. 4 shows different approximations of the instantaneous velocity profile. The instantaneous velocity signal profile 13 approximated using a first-order method and the one with a second-order method 14. FIG. 4 also shows the actual instantaneous sprint velocity signal from fusing the velocity and acceleration signals 15.

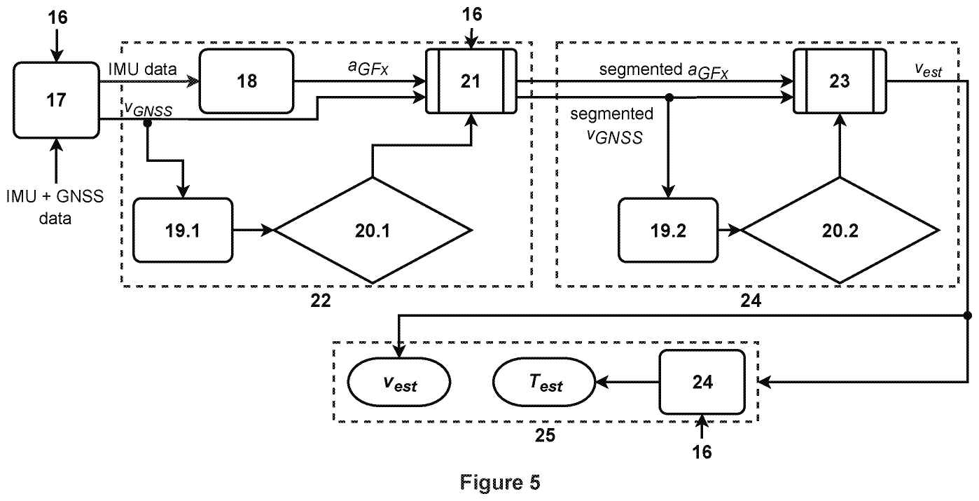

The data first recorded on the GNSS-IMU sensor (see FIG. 5) are segmented by manually selecting an approximate starting sample for the relevant sprint and entering sprint distance 16 as an input. A window 17 is created to select the approximate starting point of the relevant sprint. Acceleration signals from the segmented data are converted from runner frame of reference 5 to the global frame of reference 6 (a_{GFx}) using a gradient descent algorithm as an orientation filter 18.19.1, 20.1, 19.2, and 20.2 are used to set the noise for the GNSS velocity (v_{GNSS}) signal in the first and second phase of the algorithm respectively. A first estimation of instantaneous sprint velocity is provided 21, which is then used to segment the sprint precisely using sprint distance 16 as input. The overall sprint segmentation block 22 is represented with dotted lines. The GNSS-IMU fusion filter 23 estimates the final instantaneous velocity (vₑₛₜ) using the segmented a_{GFx} and V_{GNSS} for the sprint, within the overall velocity estimation block 24. vₑₛₜ and sprint distance 16 are given as inputs to the duration estimation block 25, which estimates the duration of the sprint (Tₑₛₜ).

The algorithm includes three phases: i) sprint segmentation 22 ii) velocity estimation 24 and iii) sprint duration estimation 25. Sprint segmentation aims to detect the period for each specific sprint. First, the data recorded on the GNSS-IMU sensor is segmented by manually selecting an approximate starting sample for the relevant sprint. Following this, the algorithm is designed to choose a precise starting time (tₛ) by selecting an appropriate threshold (0.3 m/s) on the velocity obtained from the GNSS sensor. A sensitivity analysis was conducted to see the impact of this threshold on the velocity estimation error. Acceleration signals from the segmented data are converted from the sensor frame (SF) to the global frame (GF) X-Y-Z using Equation 2:

a_{GF}=q⊗[0 a_{SF}]⊗q* (2)

Where q represents the quaternions transforming the sensor frame (SF) to the global frame (GF) and q* their transpose. These quaternions are estimated by fusing accelerometer and gyroscope data using a gradient descent algorithm (Madgwick 2011); a_{SF} is the acceleration in the sensor frame, and a_{GF} is the acceleration in the global frame X-Y-Z with positive X-axis representing the direction of sprinting.

The acceleration along the positive X-axis of the global frame (a_{GFX}) is provided as an input to the Sprint detection filter (linear Kalman filter) in combination with the ground velocity (v_{GNSS}) from the GNSS sensor. The main assumption here is that the sprinters run along a straight line (within sagittal plane), thus the acceleration a_{GFx} can be assumed to represent acceleration along the direction of running and the dynamical model of the system can be assumed to be constant. This assumption is also used during the measurements with a speed radar; in our case, it simplified the system to a linear model and allowed the use of a simple Kalman filter, which is the optimal estimator for a linear system (Burl 1998). This filter has the following prediction and update steps:

Prediction:

vₑₛₜ(n|n−1)=[1]vₑₛₜ(n−1)+[Δt]a_{GFx}(n−1)+μ (3.1)

Update:

vₑₛₜ(n|n)=vₑₛₜ(n|n−1)+K(n)(v_{GNSS}(n)−vₑₛₜ(n|n−1)) (3.2)

Kalman gain:

K(n)=p(n|n−1)(p(n|n−1)+η)⁻¹ (4)

Where vₑₛₜ is the estimated horizontal velocity, a_{GFx}(n) is the horizontal acceleration in global frame, Δt is the sampling time, μ is the process (accelerometer) noise, v_{GNSS}(n) is the velocity measured by the GNSS sensor, K(n) is the Kalman gain, p(n) is the estimation uncertainty, and η is the measurement (GNSS) noise. Since a_{GFx} has a sampling frequency of 200 Hz, v_{GNSS} is upsampled from 50 Hz to 200 Hz by ‘zero padding’. If the velocity from v_{GNSS} is non-zero, the update sequence is initiated, otherwise the prediction model continues to run without update.

The magnitudes of η and μ were set to 0.01 and 0.4 respectively, obtained via manual tuning of the filter. In order to refine the magnitude of η further, the rationale of the exponential behaviour of sprint velocity (Samozino 2016) is utilized. By subtracting both sides of equation 1 from vₘₐₓ, we get:

$${v}_{max} - {v}_{H} ( t ) = {v}_{max} ( {e}^{\{ - \frac{t}{τ} \}} ) \qquad ( 5 )$$

Based on this equation, v_{GNSS} is subtracted from the maximum velocity and an exponential curve was fitted to it and if fit is good (R²>0.91), the value of ηₖ is unchanged from 0.01. In case of a bad fit, this value is increased by an order of magnitude to 0.1. The velocity (vₑₛₜ) obtained from this Kalman filter is integrated from the starting time (tₛ) to obtain the distance profile, which is subsequently compared to the actual sprint distance and used to estimate the ending time (tₑ) and segment sprint period (t_{d}=tₑ−tₛ) precisely.

In the second phase, a more accurate exponential fitting is made using a more refined sprint period (t_{d}) obtained in the first phase. Precisely segmented v_{GNSS} and a_{GFx} are provided as inputs to the GNSS-IMU fusion filter, which is also a simple Kalman filter, with the same process and measurement models as the first filter. This filter is used to update the final sprint velocity (vₑₛₜ) precisely by considering the sprint period and the fine-tuning of GNSS noise. In the final step, vₑₛₜ is integrated to obtain the displacement-time profile and the timestamp at the relevant sprint distance is computed. The starting time (tₛ) of the sprint is then subtracted from the value of this timestamp to obtain the sprint duration (Tₑₛₜ).

To estimate force-velocity and power-velocity profiles, the first step is to estimate the approximate velocity profile from vₑₛₜ using the exponential fit (Samozino 2016) presented in equation 1. While the maximum velocity during the sprint (vₘₐₓ) and the velocity at the end (v_{end}) are the same in case of an ideal exponential velocity profile, this may not be the case with real-world velocity profiles. As a result, vₘₐₓ and v_{end} tend to deviate from each other. To investigate which velocity profile leads to a better fit, the two first-order velocity profiles, based on vₘₐₓ (v_{mdl_max,1}(t)) and v_{end} (v_{mdl_end,1}(t)) respectively, were compared to a second-order velocity profile, defined as:

v_{mdl,2}(t)=a e^{τ}^{₁}−a e^{τ}^{₂} (7)

Where τ₁, τ₂ and a were computed with the ‘trust-region reflective’ algorithm, using the ‘lsqcurvefit’ function native to Matlab application. Approximate velocity profile obtained from the best performing fitting method is differentiated to obtain the approximate horizontal acceleration a_{mdl}(t), which in combination with the sprinter's mass (M), led to the force profile:

F_{mdl}(t)=M a_{mdl}(t) (8)

Finally, the power profile was computed as a product this force profile and the velocity profile:

P_{mdl}(t)=F_{mdl}(t) a_{mdl}(t) (9)

The velocity measured at 50 Hz by the radar (v_{R}(t)) was used as reference for velocity validation. To match the sampling frequency of the reference signal, vₑₛₜ was downsampled from 200 Hz to 50 Hz by keeping the first sample and every fifth sample after the first, and V_{GNSS} was upsampled from 10 Hz to 50 Hz using linear interpolation. An error vector (equation 10) between vₑₛₜ and v_{R} was then computed for each trial. Following this, the RMS, mean, and standard deviation (SD) for each error vector were calculated. Finally, median and interquartile range (IQR) were computed for each sprint distance to investigate the bias and precision respectively. Similar procedure was applied to estimate error for V_{GNSS}.

$${ε}_{v} ( t ) = \frac{{v}_{R} ( t ) - {v}_{est} ( t )}{max ( {v}_{R} ( t ) )} \times 100 \% \qquad ( 10 )$$

In order to investigate the different fitting methods explained earlier, we calculated the error vectors (equation 11) of the fitted curves v_{mdl}(t) (i.e. v_{mdl_max,1}(t), v_{mdl_end,1}(t) and v_{mdl,2}(t)) with respect to v_{R}, followed by calculating RMS and median and IQR. Further, we also investigated the fitting performance qualitatively by observing the different fitted velocity profile curves. Similarly, the error for fitted curves with respect to vₑₛₜ was calculated.

ε_{fit}(t)=v_{R}(t)−v_{fit}(t), (11)

The time recorded in the photocells (T_{Ref}) was used as reference for validation of the estimated sprint duration (Tₑₛₜ). Percentage error for the sprint duration was calculated by equation 12:

$${ε}_{t} = \frac{{T}_{Ref} - {T}_{est}}{{T}_{Ref}} \times 100 \% \qquad ( 12 )$$

Similar process was carried out for the duration obtained from the radar (T_{rad}), in order to compare the performance of the algorithm with that of the radar. Subsequently, the RMS, median, and IQR for these error values were calculated.

Lastly, the maximum velocity is an important metric according to earlier research on sprint mechanics (J.-B. Morin 2012) and thus, we opted to compare the value obtained from our method with that from the radar. The Bland-Altman plot (mean-difference) was used (Altman 1990) for this purpose, along with the calculation of the Lin's concordance correlation coefficient (ccc) at 95% confidence interval (Lawrence 1989) as a measure of agreement between our method and the radar. A correlation coefficient value greater than 0.7 was considered ‘strong’, according to the ranges suggested in (Hopkins 2009) for sports science research. Bland-Altman plots were also utilized to compare the theoretical maximum theoretical velocity v₀ (m/s), maximum theoretical horizontal force per unit mass f₀ (N/kg), and maximum theoretical horizontal power pₘₐₓ per unit mass (W/kg) values obtained from the vₑₛₜ(t) using the second-order exponential fit to those computed from the v_{R}(t). The pₘₐₓ values were obtained from the apex values of the P-V profile.

Data for the nine athletes (7 male, 2 female, 60 m sprint time 7.39±0.37 s) was utilized in this test. Thus, a total seven sprints were considered for 30 m distance, eight for 40 m, and seventeen for 60 m. Out of these, data for two 40 m sprints was used for tuning the algorithm, while the data for all sprints was used for validation.

The median of RMS errors of the vₑₛₜ ranged from 6.2% to 8.1% (FIG. 4A, Table 1) for the three sprint distances and was lower or similar to that of the v_{GNSS} Furthermore, the IQR (Table 1) for the RMS errors for the vₑₛₜ was lower than that of the v_{GNSS}, especially for the 30 m and 60 m sprint distances.

TABLE 1
Median (IQR) values of the RMS error for v_{GNSS}, vₑₛₜ, T_{rad} and Tₑₛₜ for all three
sprint distances. RMS error was calculated on the basis of equations 10 and 12.

| Sprint | | | | | | | | |
|---|---|---|---|---|---|---|---|---|
| distance, m | % error for v_{GNSS} | | % error for vₑₛₜ | | % error for T_{rad} | | % error for Tₑₛₜ | |
| 30 | 5.6 | (4.9 to 12.0) | 6.2 | (5.2 to 7.2) | 3.3 | (1.8 to 4.5) | 0.1 | (−1.7 to 1.9) |
| 40 | 10.2 | (5.1 to 11.4) | 8.1 | (6.1 to 11.4) | −0.8 | (−2.0 to 0.2) | −4.5 | (−9.8 to 0.1) |
| 60 | 6.1 | (4.7 to 8.5) | 6.5 | (5.4 to 7.9) | −2.1 | (−3.4 to −0.2) | −6.3 | (−12.8 to −2.4) |

The median error for Tₑₛₜ ranged from 0.1% to −6.3%, while that for Tₑₛₜ varied from 3.3% to −2.3%, thus both showed a similar range. The IQR (Table 1) for T_{rad} were lower as compared to Tₑₛₜ for 40 m and 60 m sprints. For 30 m sprint, Tₑₛₜ had a lower median error, but a higher IQR than T_{rad}. For the maximum velocity (vₘₐₓ), the Bland-Altman plot showed close agreement between the estimated and the reference magnitudes, with all the values lying between the two standard deviations and the Lin's concordance correlation coefficient being 0.76 (p<0.05). The estimated values, however, showed a slight negative bias of −0.16 m/s, although this was miniscule as compared to actual maximum velocities, which are around 10 m/s. For the v₀, f₀, and pₘₐₓ the Bland-Altman plot showed close agreement between the estimated and reference values, with almost all values lying between the two standard deviations. v₀ presented a bias of −0.17 m/s which is similar to that of vₘₐₓ, f₀ showed almost zero bias, and the bias for pₘₐₓ was −0.31 W/kg, which is substantially smaller than the actual pₘₐₓ values, which range from 16 to 28 W/kg.

A qualitative presentation of the different types of exponential fits can be generated, for the first order (v_{mdl_max,1}, v_{mdl_end,1}) and second order (v_{mdl,2}) exponential fits.

For both vₑₛₜ and v_{R}, the second order fit has the lowest RMS error and lower mean and standard deviation than both first order fits (Table 2). v_{mdl_end,1} fit has similar mean error values as v_{mdl_max,1} fit for 30 m and 40 m sprints, while it has considerably higher mean value and standard deviation for the 60 m sprint (Table 2).

TABLE 2
Median (IQR) values for the RMS error in the three types of exponential fits, for all
three sprint distances. RMS error was calculated on the basis of equation 11. The second order fit
(v_{mdl, 2}) presents the lowest median (IQR) for both vₑₛₜ and v_{R}.

| Sprint | v_{mdl}_{_{—}}_{max, 1} | | v_{mdl}_{_{—}}_{end, 1} | | v_{mdl, 2} | |
|---|---|---|---|---|---|---|
| dist., m | Fit on v_{R} | Fit on vₑₛₜ | Fit on v_{R} | Fit on vₑₛₜ | Fit on v_{R} | Fit on vₑₛₜ |
| 30 | 0.53 | 0.49 | 0.51 | 0.61 | 0.34 | 0.34 |
| | (0.47 to 0.65) | (0.36 to 0.74) | (0.41 to 0.64) | (0.48 to 0.68) | (0.33 to 0.36) | (0.30 to 0.46) |
| 40 | 0.52 | 0.53 | 0.51 | 0.50 | 0.34 | 0.40 |
| | (0.46 to 0.55) | (0.32 to 0.71) | (0.41 to 0.55) | (0.36 to 0.70) | (0.31 to 0.37) | (0.26 to 0.50) |
| 60 | 0.64 | 0.51 | 1.16 | 0.47 | 0.33 | 0.35 |
| | (0.54 to 0.72) | (0.43 to 0.69) | (0.55 to 1.52) | (0.40 to 0.87) | (0.31 to 0.38) | (0.27 to 0.44) |

The sensor-fusion algorithm according to the invention can compute an accurate velocity profile with respect to the radar; it can compensate for and improve upon the accuracy of the individual IMU and GNSS velocities, as indicated in FIG. 2. The median RMS error values for the vₑₛₜ are only slightly lower than those for v_{GNSS}, whereas the standard deviation is considerably less. Thus, the velocity estimation algorithm based on GNSS and IMU fusion is robust in terms of accuracy and precision, despite the inaccuracies in the GNSS velocity.

The mean error for sprint duration (Tₑₛₜ) increased from 0.5% to −7.1% for 30 m to 60 m distances respectively, clearly showing an overestimation. This is a result of the minor underestimation of velocity caused by the residual drift in the IMU strapdown integration and the inaccuracies of the GNSS velocity.

Use of a first order exponential fit (Samozino 2016, Setuain 2018) is the dominant method of estimating the sprint velocity profile and subsequently the force (F)-power (P)-velocity (V) relationships. The accuracy of this first order exponential (equation 1) has been compared with respect to a second order exponential (equation 7) in approximating the velocity profile produced by our algorithms and by the reference radar system. FIG. 4 showed the second order fit to better approximate the velocity profile, while the first order fits led to an underestimation of the velocity. For all sprint distances, the median RMS error for second order exponential was consistently less than that for the first order exponentials; this was true for both fits based on v_{R} or vₑₛₜ. The error values are different across athletes and different sprint distances, emphasizing the idea that the velocity profile does not necessarily present first order exponential behaviour. While the first order fit is suitable to represent a maximal effort during sprint competitions (Samozino 2016), the athletes may not necessarily undertake a maximal effort during training sessions. Thus, a second order exponential can offer a truer representation of the sprinter's velocity profile across different contexts.

Use of a first order exponential leads to linear F-V and parabolic P-V profiles, which have been investigated previously (J.-B. a. Morin 2016) for their potential to predict risk of injury and to plan training goals. The second-order exponential leads to more accurate albeit non-linear F-V and non-parabolic P-V profiles. Whether the increased accuracy resulting from the second order exponential improves the analysis of athletes is a potentially important practical research question for sports scientists.

Glossary
v_{mdl}: Velocity of the exponential model
a_{mdl}: Acceleration from model
vₘₐₓ: Maximum velocity
v_{end}: Velocity at the endpoint of sprint
v_{mdl_max,1}: First order model based on maximum velocity
v_{mdl_end,1}: First order model based on speed end
v_{mdl,2}: Second order model
a_{GFx}: Forward acceleration in global frame
v_{GNSS}: Velocity measured by GNSS
vₑₛₜ: IMU-GNSS fusion estimated speed
v_{R}(t): radar speed

1. Device for measuring instantaneous sprint velocity, said device consisting of at least one position and/or one velocity sensor and one IMU sensor that respectively provide position and/or velocity and acceleration signals and wherein said signals are fused.

2. Device according to claim 1 containing at least one wearable GNSS sensor and one wearable IMU sensor that provide velocity and acceleration signals respectively, and wherein the said signals are fused according to a Kalman filter.

3. Device according to claim 1 comprising a position sensor which is a photoelectric sensor and/or ultra-wideband receiver.

4. Device according to claim 1, wherein said device is wearable.

5. Method for measuring instantaneous sprint velocity comprising the use of at least one position and/or one velocity sensor and one IMU sensor that respectively provide position and/or velocity and acceleration signals and wherein said signals are fused.

6. Method according to claim 5 comprising the use of one wearable GNSS sensor that provides a velocity signal and one wearable IMU sensor that provides an acceleration signal, wherein said signals are fused according to a Kalman filter.

7. Method according to claim 6 using furthermore a gradient descent algorithm as an orientation filter, in combination with said Kalman filter.

8. Method according to claim 7, wherein said orientation filter utilizes the IMU sensor data to convert the acceleration signals from the sensor frame to the global frame, which is then given as input to said Kalman filter for estimating the precise sprint duration.

9. Method according to claim 6 using a second order exponential model.
